(19) United States
(12) Patent Application Publication
(10) Pub. No.: US 2008/0046042 A1
(43) Pub. Date: Feb. 21, 2008

(54) Auricular therapy

(76) Inventor: Susan Jane Branch, Weymouth (GB)

Correspondence Address: JAMES C. WRAY, 1493 CHAIN BRIDGE ROAD, SUITE 300, MCLEAN, VA, 22101, US

(21) Appl. No.: 11/893,999

(22) Filed: Aug. 17, 2007

(30) Foreign Application Priority Data
Aug. 17, 2006 (GB) 06 16 345.5

(51) Int. Cl. A61N 5/06

(52) U.S. Cl. 607/88

(57) ABSTRACT

The invention is based upon the principle that stimulating a plurality of acupuncture points as will bring them to homeostatic balance. It follows that stimulating the whole pinna is as effective as stimulating individual acupuncture points. Therapeutic apparatus according to the invention comprises a pair of earpieces 10 joined by a band 12 to fit on a patient's head. Each earpiece 10 is provided with an inwardly directed light source 14 comprising a plurality of light emitting diodes (LEDs) powered from an electrical mains supply by way of a combined plug and transformer 16, a cable 18 and a control box 20. The control box 20 comprises a switch 22 whereby the dc power to the light sources 14 is turned on to irradiate the patient's outer ears with radiation of a selected wavelength and a rotary control 24 for adjusting the length of time it is on. The light source is pulsed at about 5 KHz.

### CROSS REFERENCE TO RELATED APPLICATIONS

This application claims priority from United Kingdom Patent Application No. 06 16 345.5, filed 17 Aug. 2006, the entire disclosure of which is incorporated herein by reference in its entirety.

### TECHNICAL FIELD

This invention concerns therapeutic apparatus for treating a patient—typically but not necessarily exclusively a human patient—by the application of optical radiation to the patient's pinna, which is to say the patient's outer ear or auricle.

### BACKGROUND OF THE INVENTION

Auricular therapy is a development from the traditional Chinese medicine practice of acupuncture whereby specific points on the body are stimulated to restore health and wellbeing. In auricular therapy the whole body may be treated by stimulating acupuncture points in the patient's pinna.

Traditionally, acupuncture points have been stimulated by needles, but more recently optical stimulation has been used. In U.S. Pat. No. 4,535,784, for instance, Rohlicek et al describe apparatus for stimulating an acupuncture point by focusing optical radiation (visible or infra-red) upon it, and both U.S. Pat. No. 5,250,068 (Ideguchi et al) and U.S. Pat. No. 5,843,074 (Cocilovo) describe the application of light to acupuncture points by means of optical fibres. Also, as described in U.S. Pat. No. 4,865,035, Mori applies light to a patient's ear by means of an optical conductor cable, but not particularly to acupuncture points.

It is to be noted that other prior devices provide a beam of radiation spread over the whole of a patient's pinna (or even more) but without any reference to stimulating acupuncture points. For instance, European Patent Application 1 086 673 (Fisher & Paykel) concerns a radiant heater with a beam focused on a patient's neck and ears (or other areas with a high concentration of arteriovenous anastomoses) so as to keep the patient warm during surgery. And U.S. Pat. No. 7,020,902 (Tyler) describes a headband with ear pieces holding light bulbs that radiate heat to the wearer's ears, simply for use as an ear warmer. There is no indication that such prior devices provide any substantial stimulation of acupuncture points.

In most of the prior systems for applying optical radiation to acupuncture points, the radiation is narrowly focussed onto specific acupuncture points. This was by design; as noted by Cocilovo, “When treatment of acupuncture micro-systems is preferred, such as ear acupuncture, the size of the illuminating tip is extremely important and should be as small as possible.” However, this requirement of focusing stimulation on specific acupuncture points means that auricular therapy has been costly and complex.

### BRIEF SUMMARY OF THE INVENTION

Thus according to the invention there is provided therapeutic apparatus for treating a patient by the application of optical radiation to the patient's pinna, which apparatus comprises a beam of optical radiation extending over substantially all of the patient's pinna and modulation means operable to pulse the optical radiation.

This technique is not limited to stimulating specific acupuncture points. Since acupuncture points respond to stimulation only if they need to change, stimulating a plurality of points will bring them to homeostatic balance. It follows that stimulating the whole pinna has no detrimental effects and is as effective as stimulating individual acupuncture points. Thus the invention provides means whereby a plurality of acupuncture points may be stimulated and brought to homeostatic balance.

Further, research carried out by the Applicant has shown that better therapeutic results are obtained from stimulating acupuncture points by radiation that is pulsed.

It should be noted here that the term ‘optical radiation’ as used herein is not intended to be limited to the visible range of light.

Preferably the optical radiation is pulsed at about 5 kHz. It is also preferred that the optical radiation has a rectangular waveform. It is further preferred that the optical radiation is pulsed with a duty cycle greater than 50% and more so if pulsed with a duty cycle not less than 80%.

The modulation of the optical radiation (which, it should be noted, need not be restricted to the visible range) may serve to adjust the wavelength of the radiation to tackle specific conditions. Notably, infra-red radiation, in the range 625-660 nm, may relieve addiction, anxiety and stress; it may help patients to lose weight; and it has sedative qualities which in appropriate circumstances may reduce the need for anaesthetic. However the present invention is by no means limited to infra-red radiation, and green light has been found to have an antidepressant effect, whilst white light may be used to combat seasonal affective disorder (SAD) syndrome.

Therapeutic apparatus according to the invention may comprise an earpiece which is applicable to the patient's pinna and configured and arranged to direct the beam on to the pinna. In this arrangement there may be a pair of said earpieces, one for each pinna of the patient. Alternatively the apparatus may comprise a second earpiece applicable to the patient's other pinna, which second earpiece is configured and arranged to allow the passage of sound—so that, for instance, the patient may comfortably listen to a television or radio programme while receiving treatment. The apparatus may otherwise be configured and arranged to be handheld, for application to the ear by the patient or by a therapist.

For improved safety, the apparatus may be powered from an electrical battery or from electrical mains by way of a step-down transformer.

The apparatus preferably comprises a timer operable to turn off the optical radiation after a predetermined period, whereupon the apparatus may deliver an audible signal.

The apparatus preferably also comprises a light source, such as a plurality of light emitting diodes (LEDs) for said optical radiation. Such a light source may be releasably secured to the apparatus so as to be replaceable, eg by a light source of another colour. This invention concerns therapeutic apparatus for treating a patient—typically but necessarily exclusively a human patient—by the application of optical radiation to the patient's pinna, which is to say the patient's outer ear or auricle.

### BRIEF DESCRIPTION OF THE SEVERAL VIEWS OF THE DRAWINGS

Other features of the invention will be apparent from the following description, which is made by way of example only and with reference to the accompanying schematic drawings, in which—

FIG. 1 illustrates a first form of apparatus embodying the invention;

FIG. 2 illustrates a second form of apparatus embodying the invention;

FIG. 3 illustrates a third form of apparatus embodying the invention;

FIG. 4 outlines a system block diagram for any of the forms of apparatus shown in FIGS. 1 to 3.

### DESCRIPTION OF THE BEST MODE FOR CARRYING OUT THE INVENTION

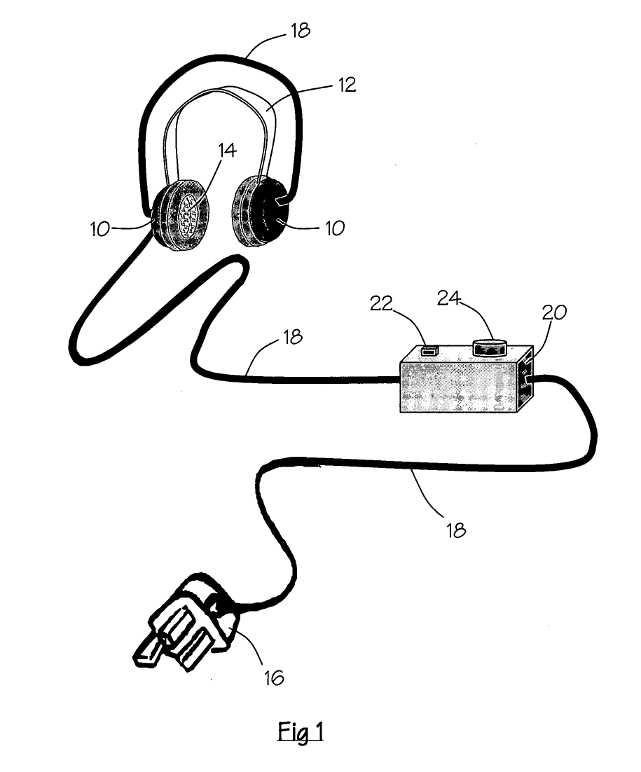

Referring first to FIG. 1, this shows therapeutic apparatus comprising a pair of earpieces 10 joined by an arcuate band 12. The earpieces 10 and the band 12 are configured and arranged so that the assembly sits comfortably on a patient's head with each earpiece 10 covering one of the patient's ears. (Although not detailed in the drawings, it will be understood that the band 12 may be adjustable in length so as to fit the assembly more comfortably to the patient's head). Each earpiece 10 is provided with an inwardly directed light source 14, of which only one is visible in FIG. 1.

Each light source 14 comprises a plurality of light emitting diodes (LEDs) powered from an electrical mains supply (not detailed) by way of a plug 16, a cable 18 and a control box 20. For safety reasons, the plug 16 includes a transformer which steps the power down to 12V dc, although those skilled in the science will appreciate that the transformer may be separate from the plug 16 and may for instance be included in the control box 20.

The control box 20 comprises a switch 22 whereby the dc power to the light sources 14 is turned on. The control box 20 also comprises a rotary control 24 connected to a timing circuit within the control box for turning the power off after a predetermined period that may be varied by means of the rotary control 24, together with an audible alarm such as a beeper to indicate when such period has ended. Within the control box 20 is additional circuitry such that the supply to the light sources 14 is a 5 kHz square wave. The timing circuit and the square wave circuitry may be of any appropriate form and it is not considered necessary to describe them in detail at this point.

The operation of the apparatus shown in FIG. 1 will now be described, first in relation to a patient for whom infra-red treatment has been selected. (In the case of self-treatment, the patient himself may make the selection with reference to instructions provided with the apparatus. Otherwise the treatment may be recommended and/or administered by a trained therapist.) In the present example, infra-red treatment, with radiation in the range 625-660 nm up to say 30 min, may be appropriate for patients suffering from stress, anxiety of addiction, or for patients wanting to lose weight, or for patients requiring sedation. For this purpose the light sources 14 will each comprise a plurality of high intensity infra-red LEDs. The earpieces 10 are applied one to each of the patient's ears (by the therapist or by the patient himself) with the band adjusted to the patient's head, then the rotary timer 24 is set to the specified time period and finally the apparatus is switched on by means of the switch 22. The patient can now rest, and perhaps doze, whilst each pinna is irradiated for the specified period, at the end of which a buzzer sounds to signal that the treatment is complete. The treatment may of course be repeated as and when required and preferably under the direction of the therapist.

High intensity white LED light sources may be used for treating SAD syndrome, and high intensity green LEDs have an antidepressant effect. The light sources 14 may plug in to the earpieces 10 so that the colours may be changed conveniently.

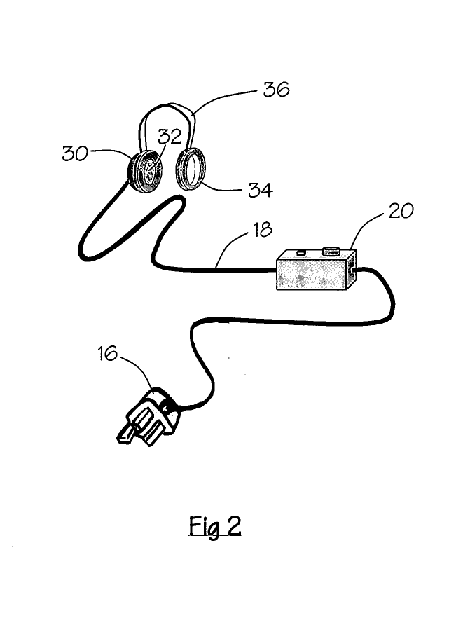

FIG. 2 shows a modification of the apparatus of FIG. 1. In the arrangement of FIG. 2 one earpiece is substantially the same as the earpiece 10 of FIG. 1 and includes a light source 32 but the other earpiece 34 simply comprises an open ring, with no light source. By this arrangement the apparatus is comfortable for the patient and the open form of the earpiece 34 means that his hearing is unimpeded, at least on one side. Thus patients who do not simply wish to rest during their treatment may instead listen to the radio or watch television. Those skilled in the science will note that the cable 18 does not extend over the top of the band 36 in the apparatus of FIG. 2, because power for the light source 32 is required on only one side.

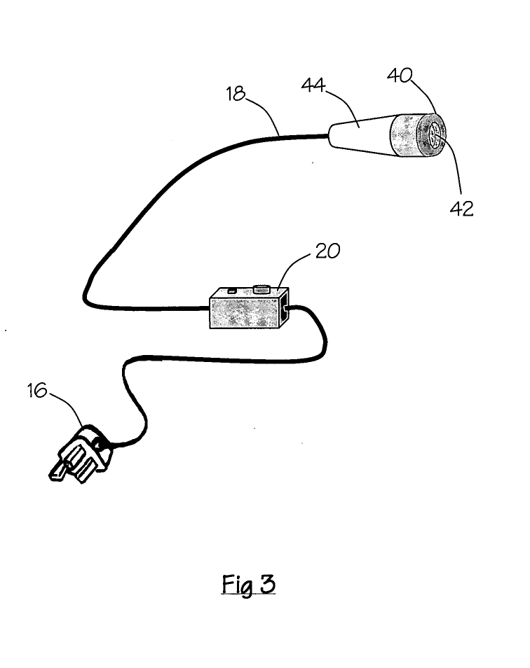

A simplified form of the apparatus is shown in FIG. 3. As shown in FIG. 3 an earpiece 40 with included light source 42 is mounted on the end of a barrel 44 designed to be handheld. With this form of the apparatus the earpiece 40 is simply pressed lightly upon the patient's pinna for the duration of the treatment.

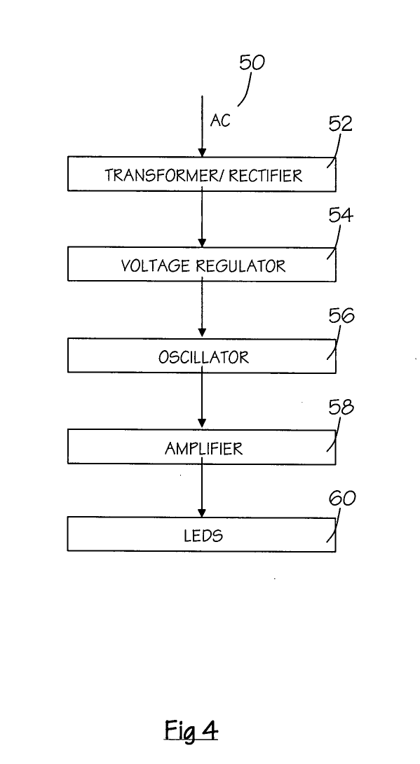

A system block diagram for the invention (which may be as embodied in FIG. 1 or FIG. 2 or FIG. 3) is shown at FIG. 4. An alternating current mains supply at 50 is fed to a transformer and rectifier 52 and then to a voltage regulator 54. The 12V output from the voltage regulator 54 powers a 5 KHz square wave oscillator 56, and the output from this is then amplified by an amplifier 58 before delivery to the LED array 69.

It is considered that those skilled in the science will be well able to implement the invention without further details.

Clinical trials of apparatus according to the invention indicate that therapeutic benefits are improved when in any treatment session the radiation is square wave pulsed with a duty cycle of 50% or above (ie so that the pulse duration is not less than 50% of the pulse period) and the benefits are most pronounced when the duty cycle is not less than 80%. This can be achieved by adjusting the square wave generator in well known fashion.

The invention has been found to be of particular benefit in treating obesity or otherwise stimulating weight loss. Causes of obesity are manifold and varied: examples are hypothyroidism, sluggish digestion, lack of exercise, overeating (which may in turn be due to poor gastric tone), low blood sugar, fluctuating blood sugar and comfort eating. With so many different possible causes it is difficult to identify the key factors in the case of any individual patient. But the invention effectively allows the patient's body to decide how to respond, rather than the clinician having to make difficult decisions about causes and possible treatments. Thus use of the invention with different patients wanting to lose weight has resulted in a wide range of (appropriate) patient responses including normalising of long-term constipation, causing earlier/easier satiation when the patient eats, suppressing the patient's desire to eat between meals and reducing comfort eating by making the patient feel generally happier.

Various possible modifications and adaptations may be made to the forms of the invention described. For instance, a battery may be used to power the apparatus instead of transformed mains power. Other variations will be apparent to those skilled in the science.

1. Therapeutic apparatus for treating a patient by the application of optical radiation to the patient's pinna, which apparatus comprises a beam of optical radiation extending over substantially all of the patient's pinna and modulation means operable to pulse the optical radiation.

2. Therapeutic apparatus as claimed in claim 1 wherein the optical radiation is pulsed at about 5 kHz.

3. Therapeutic apparatus as claimed in claim 1 wherein the optical radiation has a rectangular waveform.

4. Therapeutic apparatus as claimed in claim 3 wherein the optical radiation is pulsed with a duty cycle greater than 50%.

5. Therapeutic apparatus as claimed in claim 3 wherein the optical radiation is pulsed with a duty cycle not less than 80%.

6. Therapeutic apparatus as claimed in claim 1 wherein the modulation means is operable to adjust the optical radiation to a selected wavelength.

7. Therapeutic apparatus as claimed in claim 6 wherein the selected wavelength is in the range 625-660 nm.

8. Therapeutic apparatus as claimed in claim 6 wherein the optical

9. Therapeutic apparatus as claimed in claim 6 wherein the optical radiation is of white light.

10. Therapeutic apparatus as claimed in claim 1 wherein said apparatus comprises an earpiece which is applicable to the patient's pinna and configured and arranged to direct the beam on to the pinna.

11. Therapeutic apparatus as claimed in claim 10 wherein said apparatus comprises a pair of said earpieces, one for each pinna of the patient.

12. Therapeutic apparatus as claimed in claim 10 wherein said apparatus comprises a second earpiece applicable to the patient's other pinna, which second earpiece is configured and arranged to allow the passage of sound.

13. Therapeutic apparatus as claimed in claim 10 wherein said apparatus is configured and arranged to be handheld.

14. Therapeutic apparatus as claimed in claim 1 wherein said apparatus is battery powered.

15. Therapeutic apparatus as claimed in claim 1 wherein said apparatus is powered from electrical mains by way of a step-down transformer.

16. Therapeutic apparatus as claimed in claim 1 wherein said apparatus comprises a timer operable to turn off the optical radiation after a predetermined period.

17. Therapeutic apparatus as claimed in claim 16 wherein said apparatus delivers an audible signal at the end of said predetermined period.

18. Therapeutic apparatus as claimed in claim 1 wherein said apparatus comprises a light source for said optical radiation.

19. Therapeutic apparatus as claimed in claim 18 wherein the light source is releasably secured to the apparatus so as to be replaceable.

20. Therapeutic apparatus as claimed in claim 18 wherein the light source comprises a plurality of LEDs.
